(19) United States
(12) Patent Application Publication
(10) Pub. No.: US 2023/0200882 A1
(43) Pub. Date: Jun. 29, 2023

(54) PERICARDIOTOMY DEVICES AND RELATED METHODS

(71) Applicant: AtriCure, Inc., Mason, OH (US)

(72) Inventors: Christopher Widenhouse, Mason, OH (US); Jacob Luisi, Cincinnati, OH (US); Rachel Lauren Budke, Cincinnati, OH (US); Christoph Lewis Gillum, Maineville, OH (US)

(73) Assignee: AtriCure, Inc., Mason, OH (US)

(21) Appl. No.: 17/588,227

(22) Filed: Jan. 29, 2022

Related U.S. Application Data
(60) Provisional application No. 63/294,455, filed on Dec. 29, 2021.

(51) Int. Cl. A61B 18/14

(52) U.S. Cl. CPC A61B 18/14; A61B 2018/00351

(57) ABSTRACT

Pericardiotomy devices are disclosed. An example pericardiotomy device includes an elongated shaft and an end effector disposed distally on the shaft. The end effector includes a tip portion and a penetrating element. The tip portion includes an opening configured to engage a target portion of a pericardium and is configured, upon application of vacuum to the tip portion, to separate the target portion of the pericardium from an external surface of a heart. The penetrating element is disposed within the tip portion so that, with vacuum applied to the tip portion, the penetrating element is operative to create an opening in the target portion of the pericardium.

### CROSS REFERENCE TO RELATED APPLICATIONS

This application claims the benefit of U.S. Provisional Application No. 62/294,455, filed Dec. 29, 2021, which is incorporated by reference.

### INTRODUCTION TO THE INVENTION

The present disclosure is directed to medical instruments and devices and related methods, and, more specifically, to surgical devices for creating an opening in a pericardium, and related methods.

The present disclosure contemplates that some internal anatomic structures may be at least partially covered by one or more layers of tissue. For example, in mammals, the heart is partially surrounded by a sac-like tissue called the pericardium.

Further, the present disclosure contemplates that in connection with some surgical procedures, such as minimally invasive procedures (e.g., endoscopic procedures), it may be necessary to obtain access to such anatomic structures. For example, in connection with some cardiac procedures, it may be necessary to penetrate the pericardium to allow surgical instruments to directly access the heart. As used herein, “pericardiotomy” may refer to a surgical procedure in which an opening is created through a patient's pericardium. A pericardiotomy may be performed to allow a surgeon to access the patient's heart, such as in connection with cardiac ablation to treat atrial fibrillation and/or occlusion of the left atrial appendage.

The present disclosure contemplates that surgeons may encounter challenges when performing pericardiotomies, especially in connection with minimally invasive procedures. For example, it is generally desirable to create an opening through the pericardium without substantially affecting (e.g., cutting or burning) the underlying epicardium (i.e., the heart wall). Additionally, when a pericardiotomy is performed with the patient's heart beating (e.g., not on cardiopulmonary bypass), the movement of the heart and/or the pericardium may increase the complexity of the procedure.

While known devices and techniques have been used to perform pericardiotomies, improvements in the construction and operation of pericardiotomy devices are beneficial for users (e.g., surgeons) and patients. The present disclosure includes various improvements which may enhance the construction, operation, and methods of use of pericardiotomy devices.

It is an aspect of the present disclosure to provide a pericardiotomy device configured to create an opening through a pericardium. The pericardiotomy device may include an elongated shaft and an end effector disposed distally on the shaft. The end effector may include a tip portion including an opening configured to engage a target portion of a pericardium. The tip portion may be configured, upon application of vacuum to the tip portion, to separate the target portion of the pericardium from an external surface of a heart. The end effector may include at least one penetrating element disposed within the tip portion so that, with vacuum applied to the tip portion, the at least one penetrating element may be operative to create an opening in the target portion of the pericardium.

In a detailed embodiment, the penetrating element may be disposed at a fixed position within the tip portion. The opening may include a distal opening and/or the penetrating element may be recessed proximally within the tip portion relative to the distal opening. The penetrating element may include a generally triangular blade and/or the blade may be generally diametrically oriented within the tip portion and/or may include a distally oriented, sharpened point. The blade may include at least one diagonally oriented cutting edge. The pericardiotomy device may include an externally visible indicium indicating an orientation of the cutting edge of the blade.

In a detailed embodiment, the penetrating element may be movably disposed relative to the tip portion. The penetrating element may be distally movably disposed relative to the tip portion. The penetrating element may include a needle. The pericardiotomy device may include a handle disposed proximally on the shaft and/or the handle may include a needle-releasing actuator operable to release the needle from a retracted, proximal position into a distal, extended position. The handle may include a needle-retracting actuator operable to move the needle from the extended position to the retracted position. The handle may include a needle-depth actuator operable to adjust a longitudinal position of the needle's extended position. In some embodiments, the needle may include a hollow needle.

In a detailed embodiment, the penetrating element may be rotatably movably disposed relative to the tip portion. The penetrating element may include a needle. The pericardiotomy device may include a handle disposed proximally on the shaft and/or the handle may include a needle-rotating actuator operable to rotate the needle from a retracted, proximal position into a distal, extended position. The end effector may include a needle-operating mechanism, which may include a needle mount rotatably disposed on an axle. The needle may be disposed on the needle mount. The pericardiotomy device may include a linkage operatively coupling the needle-rotating actuator and the needle-operating mechanism. In some embodiments, the needle may include a curved needle.

In a detailed embodiment, the tip portion may be formed in a bell shape so that a proximal portion of the tip portion has an outer diameter approximately the same as an outer diameter of the shaft and a distal end of the tip portion has an outer diameter that is greater than the outer diameter of the shaft. In some embodiments, the outer diameter of the distal end of the tip portion may be about twice the outer diameter of the shaft.

In a detailed embodiment, the pericardiotomy device may include at least one vacuum connector fluidically coupled to the tip portion and configured to fluidically connect to a vacuum source.

It is an aspect of the present disclosure to provide a method of creating an opening through a pericardium, including applying vacuum to an opening of a tip portion of an end effector of a pericardiotomy device, where the end effector includes a penetrating element disposed proximate the opening, while the end effector is proximate a pericardium to draw the pericardium toward the opening; increasing a distance between a target portion of the pericardium and an exterior surface of a heart to effectuate a penetration spacing by applying vacuum to the tip portion; and/or penetrating the target portion of the pericardium to create an opening therethrough by contacting the target portion of the pericardium with the penetrating element while the penetration spacing is maintained.

In a detailed embodiment, the penetrating element may be disposed at a fixed position relative to the tip portion. Contacting the target portion of the pericardium with the penetrating element may include drawing the target portion of the pericardium proximally into the tip portion to contact penetrating element. The method may include, after contacting the target portion of the pericardium with the penetrating element, enlarging the opening. Enlarging the opening may include cutting the pericardium laterally with the penetrating element by laterally moving the end effector of the pericardiotomy device relative to the pericardium. The enlarging operation may be performed while vacuum is maintained on the tip portion.

In a detailed embodiment, the penetrating element may be distally movably disposed relative to the tip portion. Contacting the target portion of the pericardium with the penetrating element may include moving the penetrating element distally relative to the tip portion to penetrate the target portion of the pericardium. Moving the penetrating element distally relative to the tip portion to penetrate the target portion of the pericardium may include releasing the penetrating element from a proximal, retracted position to move to a distal, extended position. The method may include, before moving the penetrating element distally relative to the tip portion, moving the penetrating element proximally from the extended position to the retracted position and holding the penetrating element in the retracted position. Moving the penetrating element distally relative to the tip portion may include operating a needle-releasing actuator. The method may include, before moving the penetrating element distally relative to the tip portion, operating a needle-depth actuator to adjust a longitudinal position of the penetrating element in the extended position.

In a detailed embodiment, the penetrating element may be rotatably movably disposed relative to the tip portion. Contacting the target portion of the pericardium with the penetrating element may include rotating the penetrating element relative to the tip portion to penetrate the target portion of the pericardium. Rotating the penetrating element relative to the tip portion to penetrate the target portion of the pericardium may include rotating the penetrating element from a proximal, retracted position to a distal, extended position. The penetrating element may include a curved needle. Rotating the penetrating element relative to the tip portion to penetrate the target portion of the pericardium may include rotating the curved needle about a diametrically oriented axle. Rotating the penetrating element relative to the tip portion to penetrate the target portion of the pericardium may include operating a needle-rotating actuator disposed on a handle portion of the pericardiotomy device.

In a detailed embodiment, rotating the penetrating element from the proximal, retracted position to the distal, extended position may include rotating the penetrating element from the proximal, retracted position to a partially extended position rotationally between the retracted position and the extended position to engage the penetrating element with the target portion of the pericardium; verifying engagement of the penetrating element with the target portion of the pericardium; and/or rotating the penetrating element from the partially extended position to the extended position to cut the target portion of the pericardium to create the opening therethrough. In a detailed embodiment, the method may include, before rotating the penetrating element from the partially extended position to the extended position, moving the pericardiotomy device proximally to further increase the distance between the target portion of the pericardium and the exterior surface of a heart.

It is an aspect of the present disclosure to provide a method of creating an opening through a pericardium, including engaging an opening of a tip portion of an end effector of a pericardiotomy device with a pericardium, the end effector comprising the tip portion and at least one penetrating element disposed proximate the tip portion; separating a target portion of the pericardium from a heart by applying vacuum to the tip portion; and/or creating an opening through the target portion of the pericardium by penetrating the target portion of the pericardium with the penetrating element.

In a detailed embodiment, the penetrating element may include a fixed penetrating element disposed in a fixed position relative to the tip portion. Penetrating the target portion of the pericardium with the penetrating element may include drawing the target portion of the pericardium into the tip portion of the end effector to contact the fixed penetrating element using vacuum. The method may include enlarging the opening by moving the end effector laterally on the pericardium.

In a detailed embodiment, the penetrating element may include a distally movable penetrating element. Penetrating the target portion of the pericardium with the penetrating element may include releasing the distally movable penetrating element from a proximal, retracted position to a distal, extended position.

In a detailed embodiment, the penetrating element may include a rotatable penetrating element. Penetrating the target portion of the pericardium with the penetrating element may include rotating the penetrating element from a proximal, retracted position to a distal, extended position.

It is an aspect of the present disclosure to provide a method of engaging tissue, including repositioning an end effector proximate to a tissue surface; longitudinally rotating a penetrator from a retracted position, not in contact with the tissue surface, to an engaged position, in contact with and extending into the tissue surface; and/or repositioning the end effector in a direction opposite the tissue surface to cause the tissue to tent.

In a detailed embodiment, the method may further include continuing to longitudinally rotate the penetrator from the engaged position to a piercing position, where the penetrator pierces the tissue. The method may further include applying vacuum to the end effector when the end effector is proximate the tissue surface. Repositioning the end effector proximate to the tissue surface may include positioning the end effector to form a seal between the end effector and the tissue surface.

### BRIEF DESCRIPTION OF THE DRAWINGS

Example embodiments are described in conjunction with the accompanying drawing figures in which:

FIG. 1 is a perspective view of an example pericardiotomy device including a fixed blade;

FIG. 2 is a detailed perspective view of a proximal portion of the pericardiotomy device of FIG. 1;

FIG. 3 is a detailed perspective view of a distal portion of the pericardiotomy device of FIG. 1;

FIG. 4 is a detailed perspective view of the distal portion of the pericardiotomy device of FIG. 1;

FIG. 5 is a simplified cutaway view of the distal portion of the pericardiotomy device of FIG. 1;

FIG. 6 is a perspective view of an alternative example pericardiotomy device including a distally movable needle;

FIG. 7 is a detailed perspective view of a proximal portion of the pericardiotomy device of FIG. 6;

FIG. 8 is a detailed perspective view of a distal portion of the pericardiotomy device of FIG. 6 with the needle in an extended position;

FIG. 9 is a detailed perspective view of the distal portion of the pericardiotomy device of FIG. 6 with the needle in a retracted position;

FIG. 10 is a detailed lateral perspective view of the distal portion of the pericardiotomy device of FIG. 6 with the needle in the extended position;

FIG. 11 is a simplified cutaway view of the distal portion of the pericardiotomy device of FIG. 6;

FIG. 12 is a simplified perspective view of a needle mechanism of the pericardiotomy device of FIG. 6;

FIG. 13 is a perspective view of an alternative example pericardiotomy device including a rotatable needle;

FIG. 14 is a detailed perspective view of a distal portion of the pericardiotomy device of FIG. 13 with the needle in a retracted position;

FIG. 15 is a detailed perspective view of the distal portion of the pericardiotomy device of FIG. 13 with the needle in an extended position;

FIG. 16 is a detailed perspective view of the distal portion of the pericardiotomy device of FIG. 13;

FIG. 17 is a simplified cutaway view of the distal portion of the pericardiotomy device of FIG. 13;

FIG. 18 is a simplified perspective view of a needle mechanism of the pericardiotomy device of FIG. 13; and

FIG. 19 is a cutaway view of the handle of the pericardiotomy device of FIG. 13; all in accordance with at least some aspects of the present disclosure.

### DETAILED DESCRIPTION

Example embodiments according to the present disclosure are described and illustrated below to encompass devices, methods, and techniques relating to medical and surgical procedures, such as for creating an opening through a pericardium. Of course, it will be apparent to those of ordinary skill in the art that the embodiments discussed below are examples and may be reconfigured without departing from the scope and spirit of the present disclosure. It is also to be understood that variations of the example embodiments contemplated by one of ordinary skill in the art shall concurrently comprise part of the instant disclosure. However, for clarity and precision, the example embodiments as discussed below may include optional steps, methods, and features that one of ordinary skill should recognize as not being a requisite to fall within the scope of the present disclosure. Unless explicitly stated otherwise, any feature or function described in connection with any example embodiment may be utilized with features or functions described in connection with other example embodiments. Repeated description of similar features and functions is omitted for brevity.

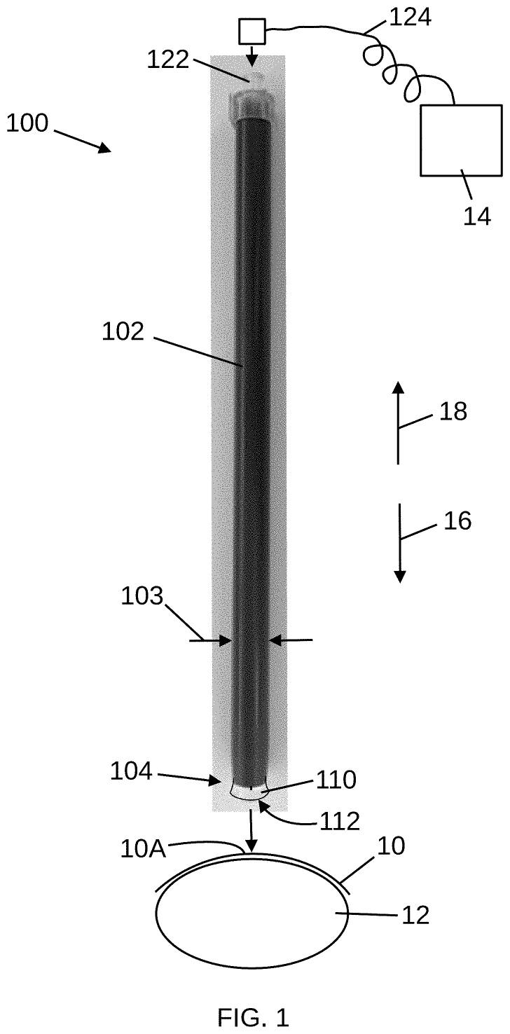

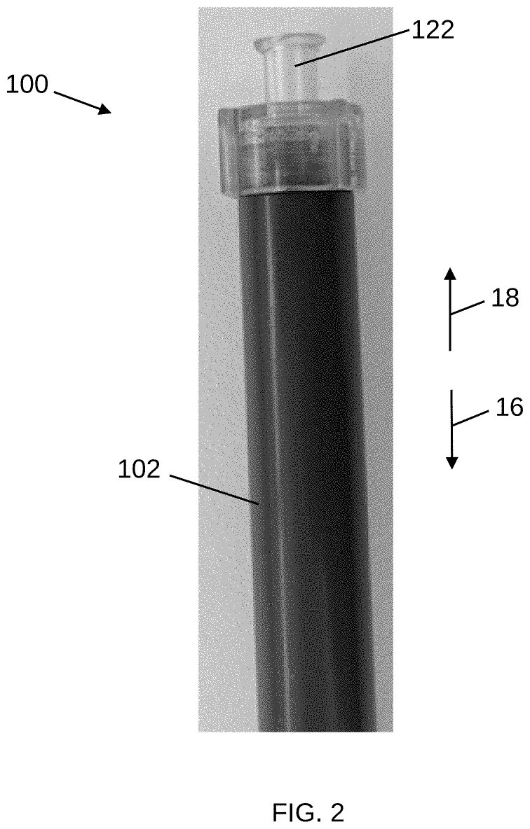

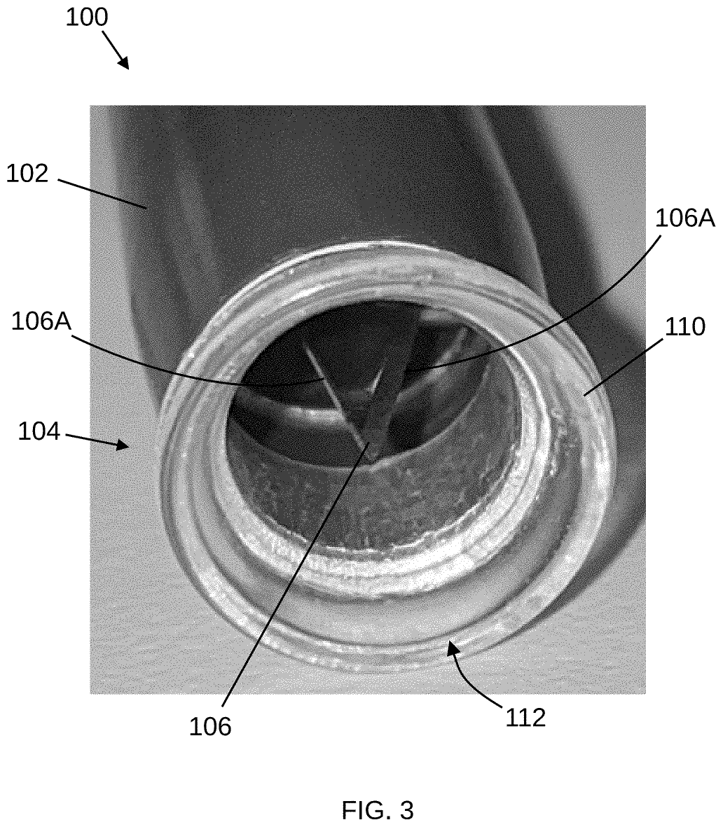

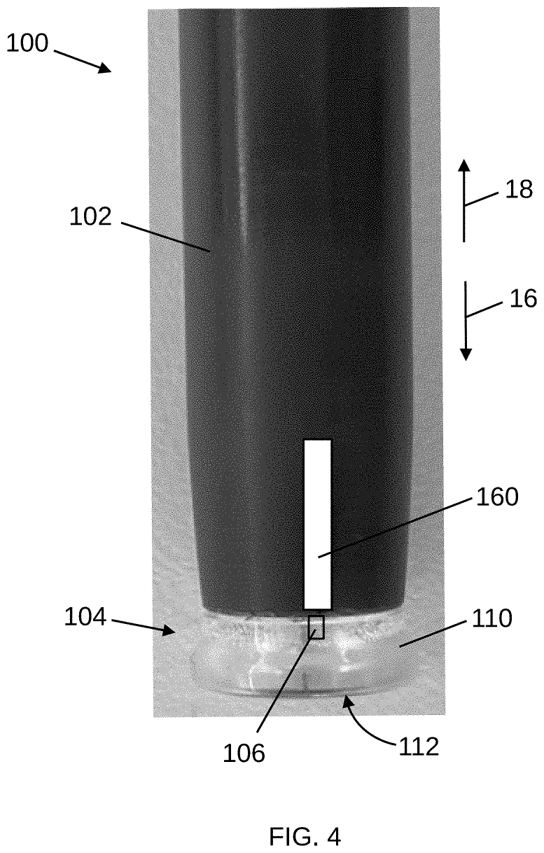

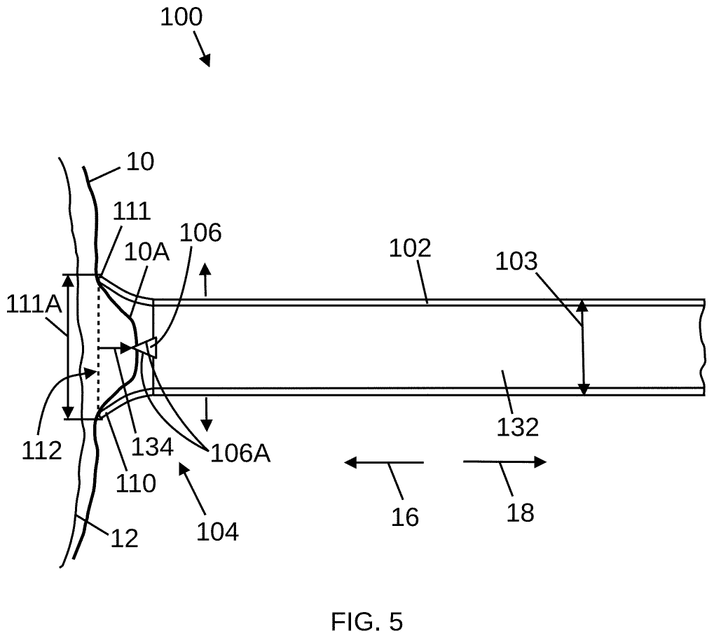

FIG. 1 is a perspective view of an example pericardiotomy device 100 including a fixed blade, FIG. 2 is a detailed perspective view of a proximal portion of the pericardiotomy device 100 of FIG. 1, FIG. 3 is a detailed perspective view of a distal portion of the pericardiotomy device 100 of FIG. 1, FIG. 4 is a detailed perspective view of the distal portion of the pericardiotomy device 100 of FIG. 1, and FIG. 5 is a simplified cutaway view of the distal portion of the pericardiotomy device 100 of FIG. 1, all according to at least some aspects of the present disclosure. Generally, the pericardiotomy device 100 may be similar in construction and operation to other pericardiotomy devices described herein, and repeated description of similar elements is omitted for brevity. Referring to FIGS. 1 and 5, the pericardiotomy device 100 may be used in connection with a pericardiotomy procedure including creating an opening through a patient's pericardium 10, such as to obtain access to the patient's heart 12. Referring to FIG. 1, the pericardiotomy device 100 may be operatively coupled to a vacuum source 14.

For clarity, the following description references a distal direction 16 and a proximal direction 18. The proximal direction 18 may be generally opposite the distal direction 16. As used herein, “distal” may refer to a direction generally away from an operator of a system or device (e.g., a surgeon), such as toward the distant-most end of a device that is inserted into a patient's body. As used herein, “proximal” may refer to a direction generally toward an operator of a system or device (e.g., a surgeon), such as away from the distant-most end of a device that is inserted into a patient's body. It will be understood, however, that example directions referenced herein are merely for purposes of explanation and clarity, and should not be considered limiting.

Referring to FIGS. 1, 3, 4, and 5, in the illustrated embodiment, the pericardiotomy device 100 includes an elongated, generally tubular shaft 102 and an end effector 104 disposed generally distally on the shaft 102. In some example embodiments, the shaft 102 may have an outer diameter 103 of about 12 mm. The shaft 102 may be configured to be substantially rigid, elastically deformable, and/or plastically deformable (when subject to forces consistent with normal, intended use of the device 100), and such characteristics may vary over the shaft's 102 proximal-distal length. In the illustrated embodiment, the shaft 102 is in the form of a tubular right circular cylinder defining an internal channel 132 extending longitudinally therethrough.

Referring to FIGS. 3-5, in the illustrated embodiment, the end effector 104 includes a tip portion 110 and a penetrating element, such as one or more blades 106 configured to create an opening through the pericardium 10. In the illustrated embodiment, the blade 106 is fixed relative to the tip portion 110. That is, the blade 106 is disposed at a fixed position relative to the tip portion 110, such as generally within the tip portion 110, and the blade 106 does not move relative to the tip portion 110 during use of the pericardiotomy device 100. The blade 106 may be disposed at least partially within the tip portion 110. In the illustrated embodiment, the blade 106 may be generally diametrically oriented. Referring to FIG. 3, in some embodiments, the blade 106 may be generally triangular in shape, with a distally oriented, sharpened point. In some embodiments, the blade 106 may include one or more cutting edges 106A, which may be oriented generally diagonally.

Referring to FIGS. 1, 3, 4 and 5, the tip portion 110 may include an opening, such as a distal opening 112, configured to engage the pericardium 10 and allow the pericardium 10 to contact the blade 106. In some example embodiments, the distal opening 112 may be generally circular. In some example embodiments, the tip portion 110 may be constructed from a substantially transparent material (e.g., a substantially optically clear material), which may facilitate visibility through the tip portion 110, such as visualization of the blade 106. In other embodiments, at least a portion of the tip portion 110 may be constructed at least partially of a translucent material and/or an opaque material, such as, without limitation, a radiopaque material.

Referring to FIGS. 1 and 2, the pericardiotomy device 100 may include at least one vacuum connector 122, which may be used to fluidically connect the pericardiotomy device 100 to the vacuum source 14. In the illustrated embodiment, the vacuum connector 122 is disposed generally proximally on the shaft 102, and the vacuum connector 122 is configured to couple with a vacuum line 124 including one or more lumens. In other embodiments, the vacuum line 124 may extend from the pericardiotomy device 100, and the vacuum connector 122 may be disposed on the end of the line 124 that attaches to the vacuum source 14. Example vacuum sources 14 include vacuum pumps and connections to central vacuum systems, such as may be available in a hospital or surgical facility.

Referring to FIGS. 1 and 5, vacuum from the vacuum source 14 may be selectively applied to the tip portion 110, such as via the internal channel 132 of the shaft 102. The channel 132 may be configured as a vacuum conduit fluidically interposing the tip portion 110 and the vacuum connector 122. Application of vacuum to the tip portion 110 (e.g., via the vacuum connector 122 and the internal channel 132) may be operative to draw a target portion 10A of the pericardium 10 into the tip portion 110 as illustrated by arrow 134. In this embodiment, drawing the target portion 10A of the pericardium 10 into the tip portion 110 by application of vacuum contacts the pericardium 10 with the blade 106. In the illustrated embodiment, the blade 106 is recessed proximally within the tip portion 110 at a distance shown by arrow 134. In other embodiments, the blade 106 may be disposed at other proximal-distal positions within the tip portion 110, such as substantially even with the distal end of the tip portion 110 (e.g., zero distance recess). In the illustrated embodiment, because the blade 106 is recessed proximally within the tip portion 110, drawing the pericardium 10 proximally into the tip portion 110 by application of vacuum also creates and/or increases the distance between the surface of the heart 12 and the location where blade 106 is used to cut through the pericardium 10. Thus, the likelihood of injury to the heart 12 (e.g., due to contact with the blade 106) may be reduced.

Referring to FIG. 5, in the illustrated embodiment, the tip portion 110 may be formed in a narrow bell shape. For example, the proximal portion of the tip portion 110 may have an outer diameter approximately the same as the outer diameter 103 of the shaft 102 (e.g., about 12 mm). The distal end 111 of the tip portion 110 may have an outer diameter 111A that is greater than the outer diameter 103 of the shaft 102 (e.g., about 13 mm). In some embodiments, the radially outer surface of the tip portion 110 may continuously curve, such as in a generally concave manner, between the proximal portion of the tip portion 110 near the shaft 102 and the distal end 111 of the tip portion 110.

Referring to FIG. 4, some example embodiments may include an externally visible indicium 160, which may be configured to indicate to a user the orientation of the pericardiotomy device 100, such as the orientation of the blade 106. In the illustrated embodiment, the indicium 160 indicates the radial direction generally aligned with the diagonal cutting edges 106A of the blade 106.

Referring to FIGS. 1 and 5, in use, the pericardiotomy device 100 may be positioned so that the distal end 111 of the tip portion 110 is in contact with the pericardium 10. Vacuum may be applied to the tip portion 110, which may draw the target portion of the pericardium 10A proximally into the tip portion 110. The target portion of the pericardium 10A may be drawn proximally into contact with the blade 106 (e.g., the distally oriented point), which may cut through the target portion of the pericardium 10A, creating an opening through the pericardium 10. If it is desired to enlarge the opening through the pericardium 10, the end effector 104 of the pericardiotomy device 100 may be moved laterally relative to the pericardium, such as while vacuum is maintained, which may cause the blade 106 to cut the pericardium 10 laterally. For example, the pericardiotomy device 100 may be moved generally in the direction indicated by the indicia 160 to cut the pericardium 10 using the cutting edge 106A of the blade 106 to create the elongated opening. The vacuum may be discontinued, and the pericardiotomy device 100 may be moved away from the target portion of the pericardium 10A.

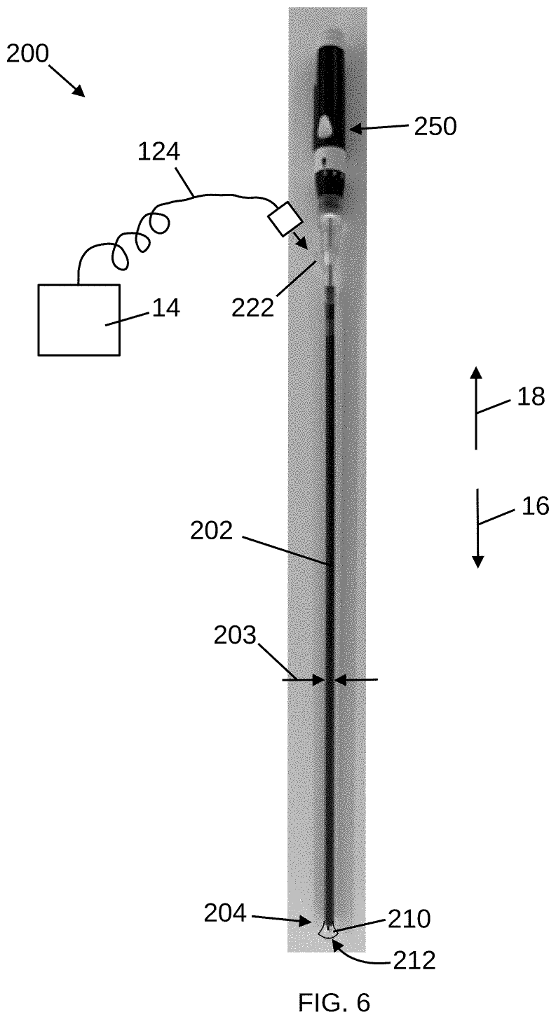

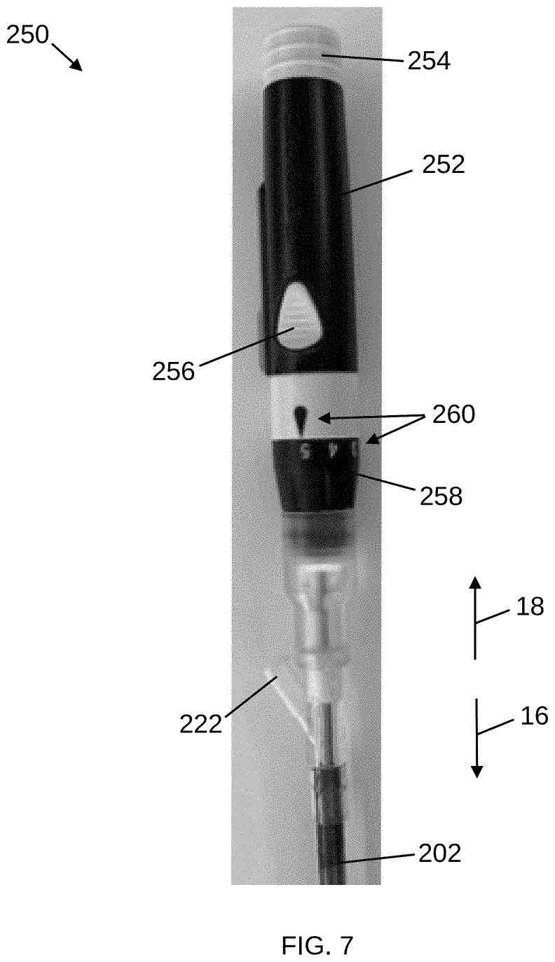

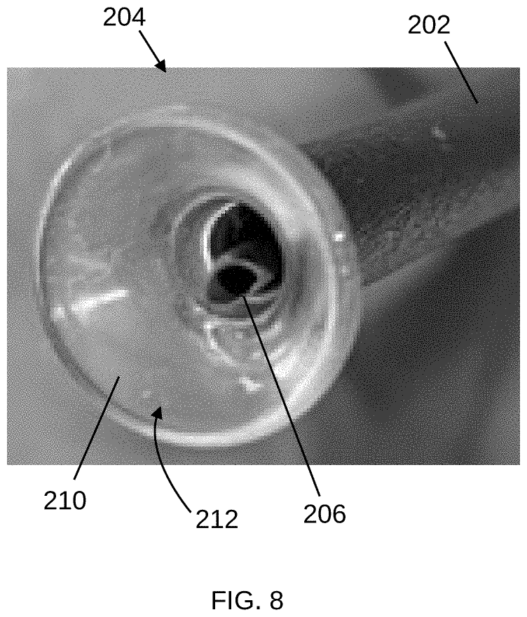

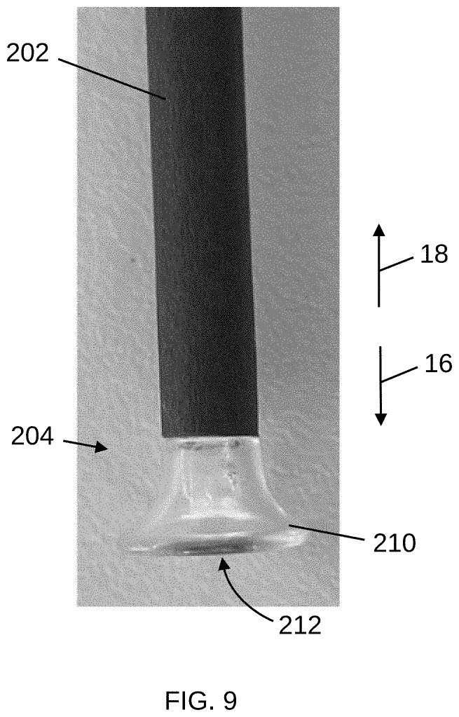

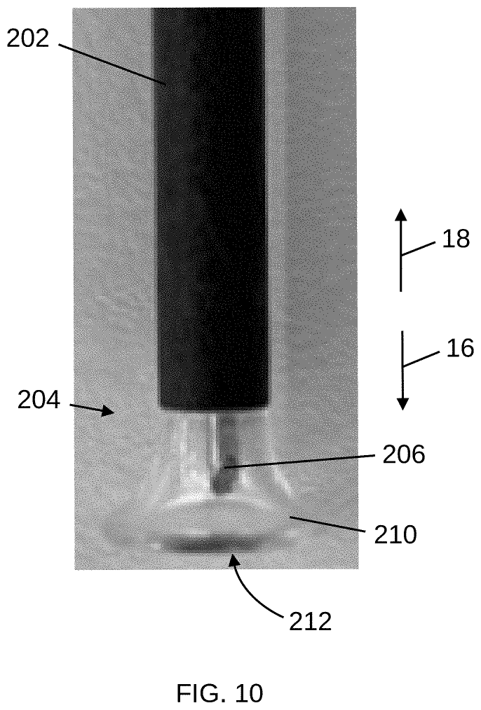

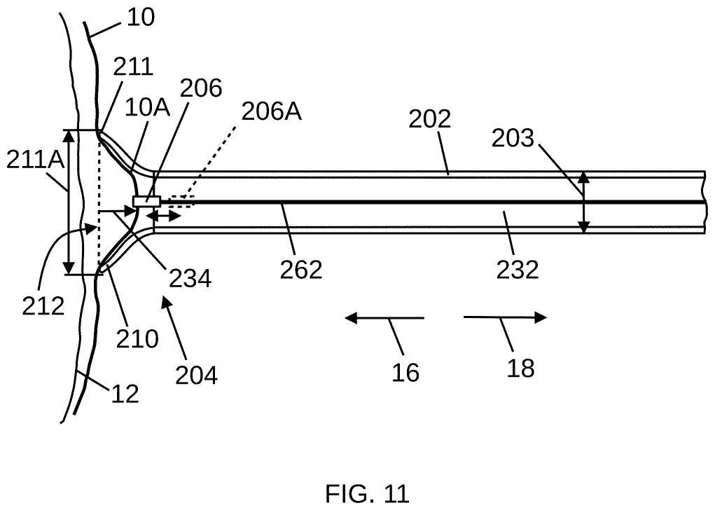

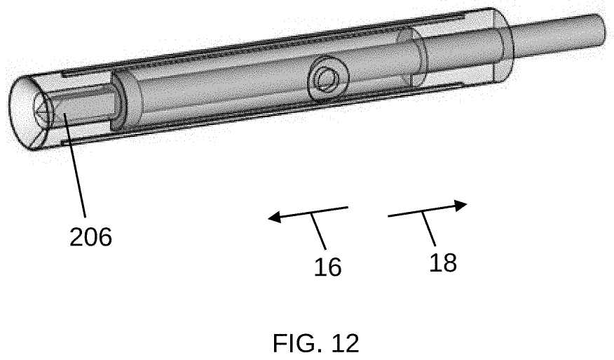

FIG. 6 is a perspective view of an alternative example pericardiotomy device 200 including a distally movable needle, FIG. 7 is a detailed perspective view of a proximal portion of the pericardiotomy device 200 of FIG. 6, FIG. 8 is a detailed perspective view of a distal portion of the pericardiotomy device 200 of FIG. 6 with the needle in an extended position, FIG. 9 is a detailed perspective view of the distal portion of the pericardiotomy device 200 of FIG. 6 with the needle in a retracted position, FIG. 10 is a detailed lateral perspective view of the distal portion of the pericardiotomy device 200 of FIG. 6 with the needle in the extended position, FIG. 11 is a simplified cutaway view of the distal portion of the pericardiotomy device 200 of FIG. 6, and FIG. 12 is a simplified perspective view of a needle mechanism of the pericardiotomy device 200 of FIG. 6, all according to at least some aspects of the present disclosure. Generally, the pericardiotomy device 200 may be similar in construction and operation to other pericardiotomy devices described herein, and repeated description of similar elements is omitted for brevity. Referring to FIG. 11, the pericardiotomy device 200 may be used in connection with a pericardiotomy procedure including creating an opening through a patient's pericardium 10, such as to obtain access to the patient's heart 12. Referring to FIG. 6, the pericardiotomy device 200 may be operatively coupled to a vacuum source 14.

Referring to FIGS. 6-11, in the illustrated embodiment, the pericardiotomy device 200 includes an elongated, generally tubular shaft 202, an end effector 204 disposed generally distally on the shaft 202, and a handle 250 disposed generally proximally on the shaft 202. In some example embodiments, the shaft 202 may have an outer diameter 203 of about 5.0 mm. The shaft 202 may be configured to be substantially rigid, elastically deformable, and/or plastically deformable (when subject to forces consistent with normal, intended use of the device 200), and such characteristics may vary over the shaft's 202 proximal-distal length. In the illustrated embodiment, the shaft 202 is in the form of a tubular right circular cylinder defining an internal channel 232 extending longitudinally therethrough.

Referring to FIGS. 8 and 11, in the illustrated embodiment, the end effector 204 includes a tip portion 210 and a penetrating element, such as one or more needles 206 configured to create an opening through the pericardium 10. The needle 206 may be movably disposed such as, without limitation, rotationally and/or longitudinally (in a proximal-distal direction) to selectively extend at least partially within the tip portion 210. Referring to FIGS. 8, 10, and 12, in some embodiments, the needle 206 may be hollow and/or may have a slanted, sharpened point.

Referring to FIGS. 6 and 8-11, the tip portion 210 may include an opening, such as a distal opening 212, configured to engage the pericardium 10 and allow the needle 206 to contact the pericardium 10. In some example embodiments, the distal opening 212 may be generally circular. In some example embodiments, the tip portion 210 may be constructed from a substantially transparent material (e.g., a substantially optically clear material), which may facilitate visibility through the tip portion 210, such as visualization of the needle 206. In other embodiments, at least a portion of the tip portion 210 may be constructed at least partially of a translucent material and/or an opaque material, such as, without limitation, a radiopaque material.

Referring to FIGS. 6 and 7, the pericardiotomy device 200 may include at least one vacuum connector 222, which may be used to fluidically connect the pericardiotomy device 200 to the vacuum source 14. In the illustrated embodiment, the vacuum connector 222 is disposed generally proximally on the shaft 202, and the vacuum connector 222 is configured to couple with a vacuum line 124 including one or more lumens. In other embodiments, the vacuum line 124 may extend from the pericardiotomy device 200, and the vacuum connector 222 may be disposed on the end of the line 124 that attaches to the vacuum source 14. Example vacuum sources 14 include vacuum pumps and connections to central vacuum systems, such as may be available in a hospital or surgical facility.

Referring to FIGS. 6 and 11, vacuum from the vacuum source 14 may be selectively applied to the tip portion 210, such as via the internal channel 232 of the shaft 202. The channel 232 may be configured as a vacuum conduit fluidically interposing the tip portion 210 and the vacuum connector 222. Application of vacuum to the tip 210 (e.g., via the vacuum connector 222 and the internal channel 232) may be operative to draw a target portion 10A of the pericardium 10 into the tip portion 210 as illustrated by arrow 234. In this embodiment, drawing the target portion 10A of the pericardium 10 into the tip portion 210 by application of vacuum pulls the pericardium 10 into the operating range of the needle 206. In the illustrated embodiment, in its distal-most, extended position, the needle 206 is recessed proximally within the tip portion 210 at a distance shown by arrow 234. In other embodiments, the needle 206 may be disposed at other proximal-distal positions within the tip portion 210, such as substantially even with the distal end of the tip portion 210 (e.g., zero distance recess). In the illustrated embodiment, because the needle 206 is recessed proximally within the tip portion 210 even in its distal-most, extended position, drawing the pericardium 10 proximally into the tip portion 210 by application of vacuum also creates and/or increases the distance between the surface of the heart 12 and the location where the needle 206 is used to pierce the pericardium 10. Thus, the likelihood of injury to the heart 12 (e.g., due to contact with the needle 206) may be reduced.

Referring to FIG. 11, in the illustrated embodiment, the tip portion 210 may be formed in a wide bell shape. For example, the proximal portion of the tip portion 210 may have an outer diameter approximately the same as the outer diameter 203 of the shaft 202 (e.g., about 5.0 mm). The distal end 211 of the tip portion 210 may have an outer diameter 211A that is substantially greater than the outer diameter 203 of the shaft 202. In some embodiments, the radial outer surface of the tip portion 210 may continuously curve, such as in a generally concave manner, between the proximal portion of the tip portion 210 near the shaft 202 and the distal end 211 of the tip portion 210. For example, the maximum outer diameter 211A of the tip portion 210 (e.g., proximate the distal end 211) may be about 10.5 mm. Thus, in some embodiments, the maximum outer diameter 211A of the tip portion 210 may be about twice the outer diameter 203 of the shaft 202.

Referring to FIGS. 6 and 7, in the illustrated embodiment, the handle 250 includes a grip portion 252, which may be configured to be grasped by a user (e.g., a surgeon). The handle 250 includes a proximally disposed needle-retracting actuator 254. The needle-retracting actuator 254 may be operated (e.g., pulled generally proximally) to move the needle 206 from the extended position (FIGS. 10 and 11) to the retracted position 206A (FIG. 9). The handle 250 includes a needle-releasing actuator 256, which may be operated to release the needle from the retracted position 206A (FIG. 11) to the extended position (FIGS. 10 and 11). In the illustrated embodiment, the needle-releasing actuator 256 includes a button disposed proximate the grip portion 252. The handle 250 includes a needle-depth actuator 258, which may be operated to adjust the longitudinal (e.g., proximal-distal) position of the needle's 206 extended position. In the illustrated embodiment, the needle-depth actuator 258 includes a generally cylindrical, rotatable element disposed concentrically with and/or distally to the grip portion 252. The handle 250 may include needle-depth indicia 260 arranged to indicate various predetermined positions of the needle-depth actuator 258. The actuators 254, 256, 258 are operatively coupled to the distal portion of the pericardiotomy device 200 (e.g., the end effector 204), such as by one or more suitable mechanical linkages 262 (FIG. 11).

In some example embodiments, the pericardiotomy device 200 may include a needle-operating mechanism, such as in the handle 250 and/or in the end effector 204, which may be configured to hold the needle 206 in the retracted position and/or to extend the needle 206 upon operation of the needle-releasing actuator 256. Generally, the needle 206 may be placed into the retracted position by operating the needle-retracting actuator 254. In some example embodiments, retracting the needle 206 may elastically deform a spring forming part of the needle-operating mechanism. The needle 206 may remain in the retracted position (with the spring elastically deformed) until the needle-releasing actuator 256 is operated. When the needle-releasing actuator 256 is operated, the needle 206 may be released, and the spring may drive the needle 206 into the extended position.

In use, the pericardiotomy device 200 may be prepared for use by operating the needle-retracting actuator 254 to move the needle 206 from the extended position to the retracted position 206A. The needle-depth actuator 258 may be operated to select the desired longitudinal position of the needle's 206 extended position. The pericardiotomy device 200 may be positioned so that the distal end 211 of the tip portion 210 is in contact with the pericardium 10. Vacuum may be applied to the tip portion 210, which may draw the target portion of the pericardium 10A proximally into the tip portion 210. The needle-releasing actuator 256 may be operated to release the needle from the retracted position 206A to the extended position, which may cause the needle to penetrate the target portion of the pericardium 10A, creating an opening through the pericardium 10. The vacuum may be discontinued, and the pericardiotomy device 200 may be moved away from the target portion of the pericardium 10A.

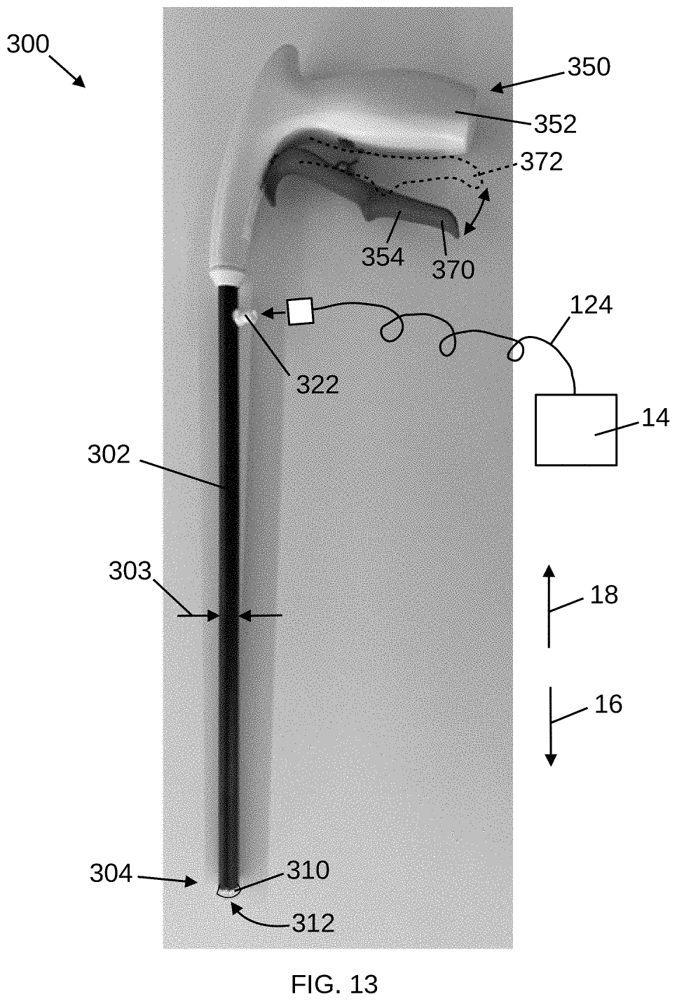

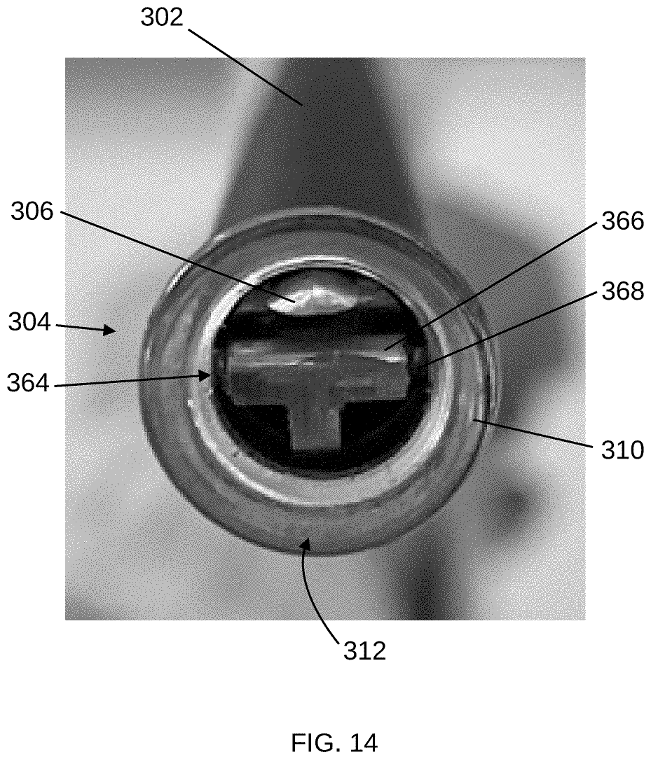

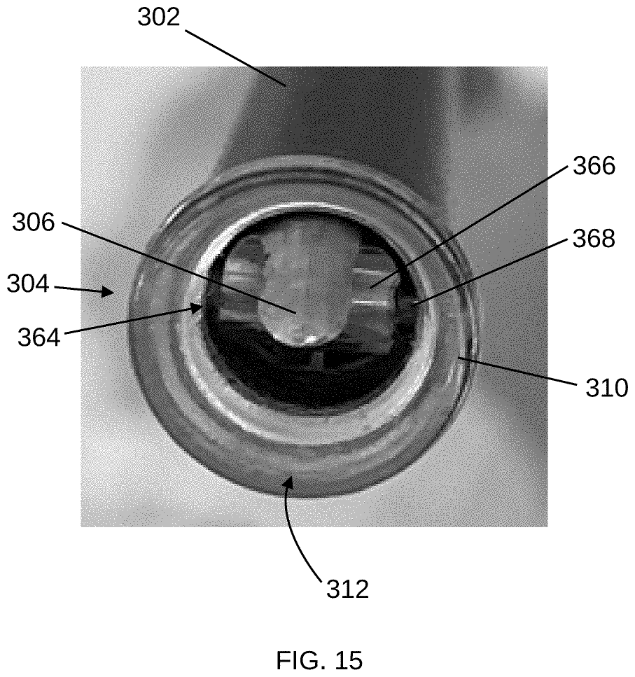

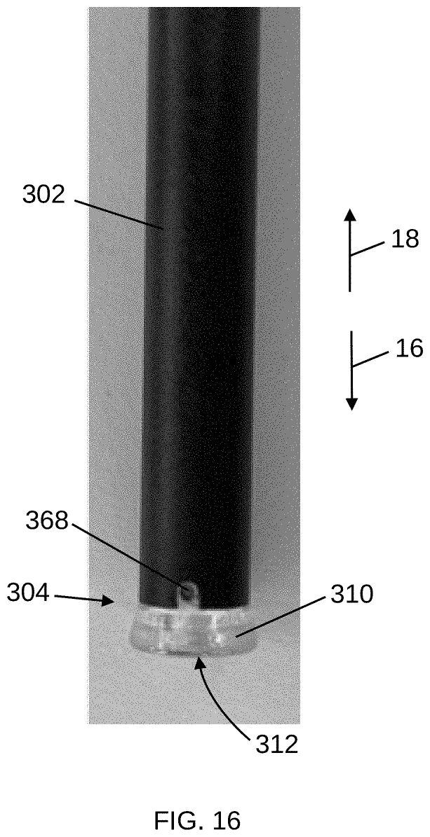

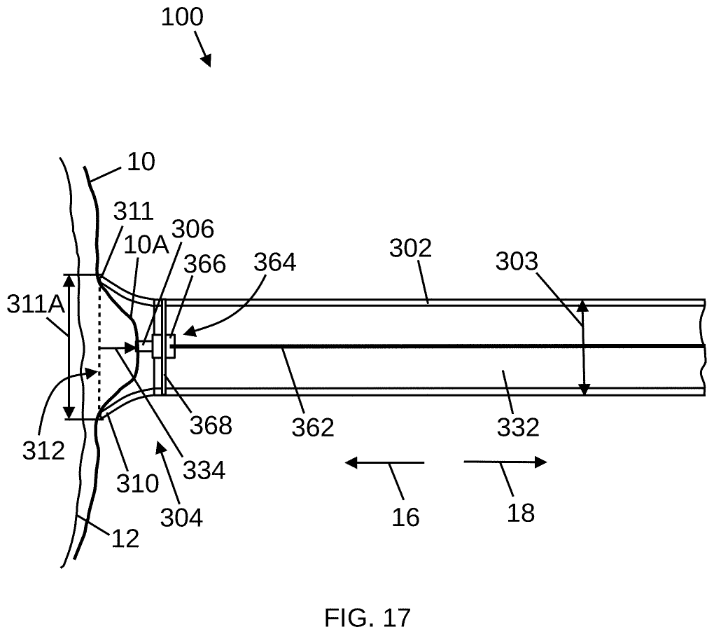

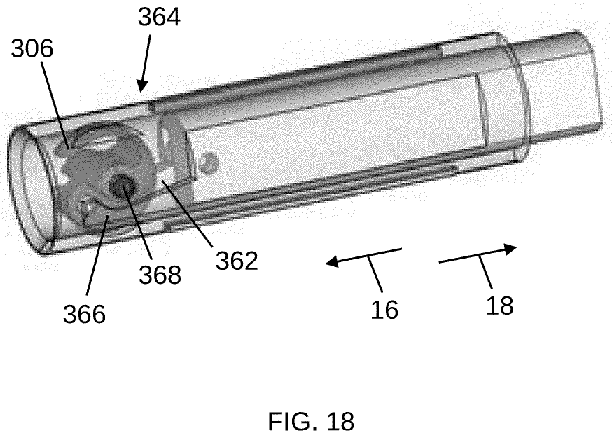

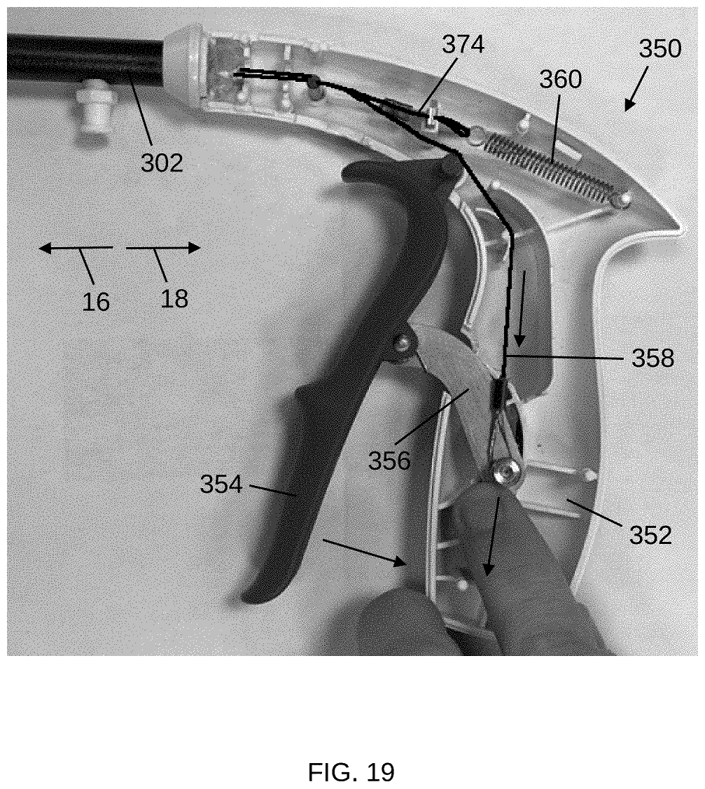

FIG. 13 is a perspective view of an alternative example pericardiotomy device 300 including a rotatable needle, FIG. 14 is a detailed perspective view of a distal portion of the pericardiotomy device 300 of FIG. 13 with the needle 306 in a retracted position, FIG. 15 is a detailed perspective view of the distal portion of the pericardiotomy device 300 of FIG. 13 with the needle 306 in an extended position, FIG. 16 is a detailed perspective view of the distal portion of the pericardiotomy device 300 of FIG. 13, FIG. 17 is a simplified cutaway view of the distal portion of the pericardiotomy device 300 of FIG. 13, FIG. 18 is a simplified perspective view of a needle mechanism of the pericardiotomy device 300 of FIG. 13, and FIG. 19 is a cutaway view of the handle of the pericardiotomy device 300 of FIG. 13, all according to at least some aspects of the present disclosure. Generally, the pericardiotomy device 300 may be similar in construction and operation to other pericardiotomy devices described herein, and repeated description of similar elements is omitted for brevity. Referring to FIG. 17, the pericardiotomy device 300 may be used in connection with a pericardiotomy procedure including creating an opening through a patient's pericardium 10, such as to obtain access to the patient's heart 12. Referring to FIG. 13, the pericardiotomy device 300 may be operatively coupled to a vacuum source 14.

Referring to FIGS. 13-17, in the illustrated embodiment, the pericardiotomy device 300 includes an elongated, generally tubular shaft 302, an end effector 304 disposed generally distally on the shaft 302, and a handle 350 disposed generally proximally on the shaft 302. In some example embodiments, the shaft 302 may have an outer diameter 303 of about 10.0 mm. The shaft 302 may be configured to be substantially rigid, elastically deformable, and/or plastically deformable (when subject to forces consistent with normal, intended use of the device 300), and such characteristics may vary over the shaft's 302 proximal-distal length. In the illustrated embodiment, the shaft 302 is in the form of a tubular right circular cylinder defining an internal channel 332 extending longitudinally therethrough.

Referring to FIGS. 14, 15, and 17, in the illustrated embodiment, the end effector 304 includes a tip portion 310 and a penetrating element, such as one or more needles 306 configured to create an opening through the pericardium 10. The needle 306 may be movably disposed longitudinally and/or radially to allow selective longitudinal rotation at least partially within the tip portion 310. In some embodiments, the needle 306 may be curved and/or may have a slanted, sharpened point. In alternative embodiments, the needle 306 may be movable and/or repositionable with respect to the tip portion 310 in any direction and/or about any axis. For example, the needle 306 may be rotatable about a longitudinally oriented axis.

The tip portion 310 may include an opening, such as a distal opening 312, configured to engage the pericardium 10 and allow the needle 306 to contact the pericardium 10. In some example embodiments, the distal opening 312 may be generally circular. In some example embodiments, the tip portion 310 may be constructed from a substantially transparent material (e.g., a substantially optically clear material), which may facilitate visibility through the tip portion 310, such as visualization of the needle 306. In other embodiments, at least a portion of the tip portion 310 may be constructed at least partially of a translucent material and/or an opaque material, such as, without limitation, a radiopaque material.

Referring to FIG. 13, the pericardiotomy device 300 may include at least one vacuum connector 322, which may be used to fluidically connect the pericardiotomy device 300 to the vacuum source 14. In the illustrated embodiment, the vacuum connector 322 is disposed generally proximally on the shaft 302, and the vacuum connector 322 is configured to couple with a vacuum line 124 including one or more lumens. In other embodiments, the vacuum line 124 may extend from the pericardiotomy device 300, and the vacuum connector 322 may be disposed on the end of the line 124 that attaches to the vacuum source 14. Example vacuum sources 14 include vacuum pumps and connections to central vacuum systems, such as may be available in a hospital or surgical facility.

Referring to FIGS. 13 and 17, vacuum from the vacuum source 14 may be selectively applied to the tip portion 310, such as via the internal channel 332 of the shaft 302. The channel 332 may be configured as a vacuum conduit fluidically interposing the tip portion 310 and the vacuum connector 322. Application of vacuum to the tip 310 (e.g., via the vacuum connector 322 and the internal channel 332) may be operative to draw a target portion 10A of the pericardium 10 into the tip portion 310 as illustrated by arrow 334. In this embodiment, drawing the target portion 10A of the pericardium 10 into the tip portion 310 by application of vacuum pulls the pericardium 10 into the operating range of the needle 306. In the illustrated embodiment, in its distal-most, extended position, the needle 306 is recessed proximally within the tip portion 310 at a distance shown by arrow 334. In other embodiments, the needle 306 may be disposed at other proximal-distal positions within the tip portion 310, such as substantially even with the distal end of the tip portion 310 (e.g., zero distance recess). In the illustrated embodiment, because the needle 306 is recessed proximally within the tip portion 310 even in its distal-most, extended position, drawing the pericardium 10 proximally into the tip portion 310 by application of vacuum also creates and/or increases the distance between the surface of the heart 12 and the location where the needle 306 is used to pierce the pericardium 10. Thus, the likelihood of injury to the heart 12 (e.g., due to contact with the needle 306) may be reduced.

Referring to FIG. 17, in the illustrated embodiment, the tip portion 310 may be formed in a relatively narrow bell shape. For example, the proximal portion of the tip portion 310 may have an outer diameter approximately the same as the outer diameter 303 of the shaft 302 (e.g., about 10.0 mm). The distal end 311 of the tip portion 310 may have an outer diameter 311A that is greater than the outer diameter 303 of the shaft 302. In some embodiments, the radial outer surface of the tip portion 310 may continuously curve, such as in a generally concave manner, between the proximal portion of the tip portion 310 near the shaft 302 and the distal end 311 of the tip portion 310. For example, the maximum outer diameter 311A of the tip portion 310 (e.g., proximate the distal end 311) may be about 11.5 mm.

Referring to FIG. 13, in the illustrated embodiment, the handle 350 includes a grip portion 352, which may be configured to be grasped by a user (e.g., a surgeon). The handle 350 includes a proximally disposed needle-rotating actuator 354. The needle-rotating actuator 354 may be operated (e.g., pulled generally proximally) to longitudinally rotate the needle 306 from the retracted position (FIG. 14) to the extended position (FIG. 15). The actuator 354 is operatively coupled to the distal portion of the pericardiotomy device 300 (e.g., the end effector 304), such as by one or more suitable mechanical linkages 362 (FIG. 17).

Referring to FIGS. 13-15, and 17-19, in some example embodiments, the pericardiotomy device 300 may include a needle-operating mechanism 364, such as in the end effector 304, which may be configured to longitudinally extend/retract and/or rotate the needle 306 upon operation of the needle-rotating actuator 354. Generally, the needle 306 may be disposed on a mount 366, which may be rotatably disposed on diametrically oriented axle 368. Operation of the needle-rotating actuator 354 on the handle 350 may be operative to rotate the mount 366 and the needle 306 about the axle 368.

In the illustrated embodiment, the needle 306 may be in the retracted position when the needle-rotating actuator 354 is in an extended position 370. Squeezing the needle-rotating actuator 354 toward the grip portion 352 into a depressed position 372 may be operative to rotate the mount 366 and the needle 306 into the extended position. For example, in the illustrated embodiment, squeezing the needle-rotating actuator 354 toward the grip portion 352 is operative to move an actuator arm 356 generally downward as illustrated in FIG. 19. Moving the actuator arm 356 downward (as shown in FIG. 19 via the arrows) pulls a cable 358 comprising a portion of the linkage 362 generally proximally, thereby causing rotation of the mount 366 and the needle 306 into the extended position (see FIG. 18). Releasing the needle-rotating actuator 354 so that it can move away from the grip portion 352 of the handle 350 allows distal movement of the cable 358. An extension spring 360 is coupled to a second cable 374, which comprises a portion of the linkage 362. The second cable 374 is operatively coupled to the rotatable mount 366 so that rotation from the retracted position to the extended position causes the cable 374 to move distally, thereby extending the spring 360. When the needle-rotating actuator 354 is released, the spring 360 pulls the second cable 374 proximally, rotating the mount 366 and needle 306 into the retracted position, pulling the cable 358 distally, and moving the actuator arm 356 and needle-rotating actuator 354 into the retracted position. Generally, in the extended position, the needle 306 may extend farther distally than when the needle is in the retracted position.

In use, the pericardiotomy device 300 may be positioned so that the distal end 311 of the tip portion 310 is in contact with the pericardium 10. Vacuum may be applied to the tip portion 310, which may draw the target portion of the pericardium 10A proximally into the tip portion 310. The needle-rotating actuator 354 may be operated to longitudinally rotate the mount 366 and the needle 306 into the extended position, which may penetrate the target portion of the pericardium 10A, creating an opening through the pericardium 10. The vacuum may be discontinued, and the pericardiotomy device 300 may be moved away from the target portion of the pericardium 10A.

Some example embodiments may be configured for multi-step longitudinal rotation of the needle 306. For example, after applying vacuum to the tip portion 310 and drawing the target portion of the pericardium 10A proximally into the tip portion 310, the needle 306 may be rotated from the retracted position to a partially extended position (e.g., rotationally between the retracted position and the fully extended position). In the partially extended position, the needle 306 may engage and/or hold the pericardium 10 (e.g., pierce the pericardium 10) without fully cutting through the pericardium 10. Accordingly, with the needle 306 in the partially extended position, the user may confirm the location where the pericardium has been engaged and/or may pull the pericardiotomy device 300 proximally to further tent the pericardium 10 away from the heart 12. If the positioning and/or engagement with the pericardium 10 are not satisfactory, the needle 306 may be returned to the retracted position, thereby releasing the pericardium 10. If the positioning and/or engagement with the pericardium 10 are satisfactory, the needle 306 may be rotated from the partially extended position to the fully extended position, thereby cutting the pericardium 10 to create the opening through the pericardium 10. Similarly, in some embodiments, movement and/or rotation of the needle 306 may be used to enlarge an opening, such as by cutting the tissue to create an elongated and/or generally circular opening.

Example methods of creating an opening through a pericardium 10 according to at least some aspects of the present disclosure may include one or more of the following operations, in any combination. A method of creating an opening through a pericardium 10 may include applying vacuum to an opening 112, 212, 312 of a tip portion 110, 210, 310 of an end effector 104, 204, 304 of a pericardiotomy device 100, 200, 300 while the end effector 104, 204, 304 is proximate a pericardium 10 to draw the pericardium 10 toward the opening 112, 212, 312. The end effector 104, 204, 304 may include a penetrating element 106, 206, 306 disposed proximate the opening 112, 212, 312. A distance 134, 234, 334 between a target portion 10A of the pericardium 10 and an exterior surface of a heart 12 may be increased to effectuate a penetration spacing by applying vacuum to the tip portion 110, 210, 310. The target portion 10A of the pericardium 10 may be penetrated to create an opening therethrough by contacting the target portion 10A of the pericardium 10 with the penetrating element 106, 206, 306 while the penetration spacing is maintained.

In some embodiments, the penetrating element 106 may be disposed at a fixed position relative to the tip portion 110. Contacting the target portion 10A of the pericardium 10 with the penetrating element 106 may include drawing the target portion 10A of the pericardium 10 proximally into the tip portion 110 to contact penetrating element 106. After contacting the target portion 10A of the pericardium 10 with the penetrating element 106, the opening may be enlarged. Enlarging the opening may include cutting the pericardium 10 laterally with the penetrating element 106 by laterally moving the end effector 104 of the pericardiotomy device 100 relative to the pericardium 10. The enlarging operation may be performed while vacuum is maintained on the tip portion 110.

In some embodiments, the penetrating element 206 may be longitudinally and/or radially repositionable relative to the tip portion 210. Contacting the target portion 10A of the pericardium 10 with the penetrating element 206 may include moving the penetrating element 206 distally relative to the tip portion 210 to penetrate the target portion 10A of the pericardium 10. Moving the penetrating element 206 distally relative to the tip portion 210 to penetrate the target portion 10A of the pericardium 10 may include releasing the penetrating element 206 from a proximal, retracted position to move to a distal, extended position. Before moving the penetrating element 206 distally relative to the tip portion 210, the penetrating element 206 may be moved proximally from the extended position to the retracted position and the penetrating element 206 may be held in the retracted position. Moving the penetrating element 206 distally relative to the tip portion 210 may include operating a needle-releasing actuator 256. Before moving the penetrating element 206 distally relative to the tip portion 210, a needle-depth actuator 258 may be operated to adjust a longitudinal position of the penetrating element 206 in the extended position.

In some embodiments, the penetrating element 306 is longitudinally rotatable relative to the tip portion 310. Contacting the target portion 10A of the pericardium 10 with the penetrating element 306 may include longitudinally rotating the penetrating element 306 relative to the tip portion 310 to penetrate the target portion 10A of the pericardium 10. Rotating the penetrating element 306 relative to the tip portion 310 to penetrate the target portion 10A of the pericardium 10 may include longitudinally rotating the penetrating element 306 from a proximal, retracted position to a distal, extended position. The penetrating element 306 may include a curved needle 306. Rotating the penetrating element 306 relative to the tip portion 310 to penetrate the target portion 10A of the pericardium 10 may include rotating the curved needle 306 about a diametrically oriented axle 3. Rotating the penetrating element 306 relative to the tip portion 310 to penetrate the target portion 10A of the pericardium 10 may include operating a needle-rotating actuator 354 disposed on a handle portion 350 of the pericardiotomy device 300.

A method of creating an opening through a pericardium 10 may include engaging an opening 112, 212, 312 of a tip portion 110, 210, 310 of an end effector 104, 204, 304 of a pericardiotomy device 100, 200, 300 with a pericardium 10. The end effector 104, 204, 304 may include the tip portion 110, 210, 310 and at least one penetrating element 106, 206, 306 disposed proximate the tip portion 110, 210, 310. A target portion 10A of the pericardium 10 may be separated from a heart 12 by applying vacuum to the tip portion 110, 210, 310. An opening through the target portion 10A of the pericardium 10 may be created by penetrating the target portion 10A of the pericardium 10 with the penetrating element 106, 206, 306.

In some embodiments, the penetrating element 106 may include a fixed penetrating element 106 disposed in a fixed position relative to the tip portion 110. Penetrating the target portion 10A of the pericardium 10 with the penetrating element 106 may include drawing the target portion 10A of the pericardium 10 into the tip portion 110 of the end effector 104 to contact the fixed penetrating element 106 using vacuum. The opening may be enlarged by moving the end effector 104 laterally on the pericardium 10.

In some embodiments, the penetrating element 206 may include a distally movable penetrating element 206. Penetrating the target portion 10A of the pericardium 10 with the penetrating element 206 may include releasing the distally movable penetrating element 206 from a proximal, retracted position to a distal, extended position.

In some embodiments, the penetrating element 306 comprises a rotatable penetrating element 306. Penetrating the target portion 10A of the pericardium 10 with the penetrating element 306 may include rotating the penetrating element 306 from a proximal, retracted position to a distal, extended position.

A method of engaging tissue may include repositioning an end effector 304 proximate to a tissue surface 10. A penetrator 306 may be longitudinally rotated from a retracted position, not in contact with the tissue surface 10, to an engaged position, in contact with and extending into the tissue surface 10. The end effector 304 may be repositioned in a direction opposite the tissue surface 10 to cause the tissue 10 to tent.

In some embodiments, the penetrator 306 may be further longitudinally rotated from the engaged position to a piercing position, where the penetrator 306 pierces the tissue 10. Vacuum may be applied to the end effector 304 when the end effector 304 is proximate the tissue surface 10. Repositioning the end effector 304 proximate to the tissue surface 10 may include positioning the end effector 304 to form a seal between the end effector 304 and the tissue surface 10.

Following from the above description and invention summaries, it should be apparent to those of ordinary skill in the art that, while the methods and apparatuses herein described constitute example embodiments according to the present disclosure, it is to be understood that the scope of the disclosure contained herein is not limited to the above precise embodiments and that changes may be made without departing from the scope as defined by the following claims. Likewise, it is to be understood that it is not necessary to meet any or all of the identified advantages or objects disclosed herein in order to fall within the scope of the claims, since inherent and/or unforeseen advantages may exist even though they may not have been explicitly discussed herein.

What is claimed is:

1. A pericardiotomy device configured to create an opening through a pericardium, the pericardiotomy device comprising:
an elongated shaft; and
an end effector disposed distally on the shaft, the end effector comprising
a tip portion comprising an opening configured to engage a target portion of a pericardium, wherein the tip portion is configured, upon application of vacuum to the tip portion, to separate the target portion of the pericardium from an external surface of a heart; and
at least one penetrating element disposed within the tip portion so that, with vacuum applied to the tip portion, the at least one penetrating element is operative to create an opening in the target portion of the pericardium.

2. The pericardiotomy device of claim 1, wherein the at least one penetrating element is disposed at a fixed position within the tip portion.

3. The pericardiotomy device of claim 2,
wherein the opening comprises a distal opening; and
wherein the at least one penetrating element is recessed proximally within the tip portion relative to the distal opening.

4. The pericardiotomy device of claim 3,
wherein the at least one penetrating element comprises a generally triangular blade; and
wherein the blade is generally diametrically oriented within the tip portion and comprises a distally oriented, sharpened point.

5-7. (canceled)

8. The pericardiotomy device of claim 1,
wherein at least one penetrating element is distally movably disposed relative to the tip portion; and
wherein the at least one penetrating element comprises a needle.

9. (canceled)

10. The pericardiotomy device of claim 8,
further comprising a handle disposed proximally on the shaft;
wherein the handle comprises a needle-releasing actuator operable to release the needle from a retracted, proximal position into a distal, extended position.

11. The pericardiotomy device of claim 10, wherein the handle comprises a needle-retracting actuator operable to move the needle from the extended position to the retracted position.

12. The pericardiotomy device of claim 10, wherein the handle comprises a needle-depth actuator operable to adjust a longitudinal position of the needle's extended position.

13. (canceled)

14. The pericardiotomy device of claim 1,
wherein at least one penetrating element is rotatably movably disposed relative to the tip portion; and
wherein the at least one penetrating element comprises a needle.

15. (canceled)

16. The pericardiotomy device of claim 14,
further comprising a handle disposed proximally on the shaft;
wherein the handle comprises a needle-rotating actuator operable to rotate the needle from a retracted, proximal position into a distal, extended position.

17-21. (canceled)

22. A method of creating an opening through a pericardium, the method comprising:
applying vacuum to an opening of a tip portion of an end effector of a pericardiotomy device, where the end effector includes a penetrating element disposed proximate the opening, while the end effector is proximate a pericardium to draw the pericardium toward the opening;
increasing a distance between a target portion of the pericardium and an exterior surface of a heart to effectuate a penetration spacing by applying vacuum to the tip portion; and
penetrating the target portion of the pericardium to create an opening therethrough by contacting the target portion of the pericardium with the penetrating element while the penetration spacing is maintained.

23. The method of claim 22,
wherein the penetrating element is disposed at a fixed position relative to the tip portion; and
wherein contacting the target portion of the pericardium with the penetrating element comprises drawing the target portion of the pericardium proximally into the tip portion to contact penetrating element.

24. The method of claim 23,
further comprising, after contacting the target portion of the pericardium with the penetrating element, enlarging the opening;
wherein enlarging the opening comprises cutting the pericardium laterally with the penetrating element by laterally moving the end effector of the pericardiotomy device relative to the pericardium.

25. (canceled)

26. The method of claim 22,
wherein the penetrating element is distally movably disposed relative to the tip portion; and
wherein contacting the target portion of the pericardium with the penetrating element comprises moving the penetrating element distally relative to the tip portion to penetrate the target portion of the pericardium.

27. The method of claim 26, wherein moving the penetrating element distally relative to the tip portion to penetrate the target portion of the pericardium comprises releasing the penetrating element from a proximal, retracted position to move to a distal, extended position.

28-29. (canceled)

30. The method of claim 27, further comprising, before moving the penetrating element distally relative to the tip portion, operating a needle-depth actuator to adjust a longitudinal position of the penetrating element in the extended position.

31. The method of claim 22,
wherein the penetrating element is rotatably movably disposed relative to the tip portion; and
wherein contacting the target portion of the pericardium with the penetrating element comprises rotating the penetrating element relative to the tip portion.

32. The method of claim 31, rotating the penetrating element relative to the tip portion comprises rotating the penetrating element from a proximal, retracted position to a distal, extended position.

33. The method of claim 32,
wherein the penetrating element comprises a curved needle; and
wherein rotating the penetrating element relative to the tip portion to penetrate the target portion of the pericardium comprises rotating the curved needle about a diametrically oriented axle.

34. (canceled)

35. The method of claim 32, wherein rotating the penetrating element from the proximal, retracted position to the distal, extended position comprises
rotating the penetrating element from the proximal, retracted position to a partially extended position rotationally between the retracted position and the extended position to engage the penetrating element with the target portion of the pericardium;
verifying engagement of the penetrating element with the target portion of the pericardium; and
rotating the penetrating element from the partially extended position to the extended position to cut the target portion of the pericardium to create the opening therethrough.

36-44. (canceled)
